(19) United States
(12) Patent Application Publication
(10) Pub. No.: US 2023/0368890 A1
(43) Pub. Date: Nov. 16, 2023

(54) A METHOD OF PREPARING A PERSONALIZED NUTRITION RECOMMENDATION FOR AN INFANT

(71) Applicant: SOCIETE DES PRODUITS NESTLE S.A., Vevey (CH)

(72) Inventors: YVONNE LENIGHAN, Linwood, NJ (US); LYNDA O’NEILL, Chatel-St-Denis (CH); SIMONA STAN, Ollon (CH); LAURENCE BIEHL, Thun (CH); ANNE M. DATTILO, Hollywood, FL (US)

(21) Appl. No.: 18/250,007

(22) PCT Filed: Oct. 20, 2021
(86) PCT No.: PCT/EP2021/079017

§ 371 Date: Apr. 21, 2023

(30) Foreign Application Priority Data
Oct. 23, 2020 (EP) 20203511.9

(51) Int. Cl. G16H 20/60; G16H 50/30

(52) U.S. Cl. CPC G16H 20/60; G16H 50/30; G16H 10/60

(57) ABSTRACT

The present invention relates generally to the field of infant nutrition. In particular, the present invention relates to a method of preparing a personalized nutrition recommendation for an infant comprising collecting information, analysing the collected information, and providing the nutrition recommendation based upon the analysed collected information; wherein the nutrition recommendation comprises a recommendation how much of a specific nutrition product for children the child should consume.

The present invention relates generally to the field of infant nutrition. In particular, the present invention relates to a method of preparing a personalized nutrition recommendation for an infant comprising collecting information, analysing the collected information, and providing the nutrition recommendation based upon the analysed collected information; wherein the nutrition recommendation comprises a recommendation how much of a specific infant nutrition product that the infant should consume.

Mother’s milk is recommended for all infants. However, in some cases breast feeding is inadequate or unsuccessful for medical reasons or the mother chooses not to breast feed. Infant formulae have been developed for these situations. Conventional infant formulae fall into two categories, the first stage formulae for infants from the age of birth to 6 months and which provide complete nutrition for this age group and the second stage formulae for infants from the age of six months to twelve months which are fed to the infants in combination with increasing amounts of other foods such as infant cereals and puréed fruits, vegetables and other foodstuffs as the process of weaning progresses.

The infant formula is usually manually prepared by measuring a quantity of warm water usually with a graduated vessel, pouring the measured water quantity in an infant formula container such as a baby bottle and adding a dosed quantity of infant formula product such as infant formula powder with a spoon or another volume dosing device and mixing (shaking, stirring, etc.) to obtain the ready-to-drink infant formula.

The choice of the infant formula product is critical and it should consider the growth profile/stage of the infant and any specific allergy requirements, digestive discomforts and/or personal diet choices. If the choice of the type of infant formula product is inappropriate, it may have negative consequences on the growth or health of the infant and may lead to nutritional deficiencies or weight issues.

Consequently, it is essential that the amount and kind of infant formula product that is administered to a child is optimally adapted to the specific life circumstances of the infant. The nutritional intake by the infant may be inconstant and be influenced by many physiological or psychological factors such as gender, growth status, appetite disturbance, the hydration level, the mood or health of the baby, etc.

Given the abundance of different nutrition products for infants that are available on the market today, it is not always easy for the parents or the caregivers to select the optimal product for a particular infant during the specific phase of development the infant is in, and considering other individual specific factors including digestive issues etc.

Hence the appropriate nutrition product for infants and the optimal amount to be administered is not always easy to determine and to implement by the parents or caregivers.

US 2006/0278093 relates to a feeding formula appliance for preparing a fluid food at a desired consumption temperature on demand including a dispenser of water and powder, comprising a data processor, memories and a user interface for inputting information necessary for preparing the infant formula. The appliance comprises a processor and a quantity detector to control dosing of the product, control the filling level and the consumption temperature and the food consumption.

Philips uGrow™ baby development tracker relates to a smartphone application which provides information as to the baby feeding and sleeping patterns and tracks his/her height and weight. The application can connect to various connected devices such as a smart bottle sleeve, a baby monitoring device, an ear thermometer, a white light ambiance system or an air purifier.

Blue Smart Mia™ connected system relates to a smartphone application providing baby’s feeding tracking service using a smart sleeve for baby bottle capable of registering feeding amount by voice recognition and registering feeding time (https://bluesmartmia.com/).

However, to the inventor’s best knowledge, no method, system or device exists today that is able to provide personalized nutrition recommendation for an infant comprising a recommendation how much of a nutrition product for infants the infant should consume.

It would therefore be desirable to have available a method, system or device that is able to provide personalized nutrition recommendation for an infant comprising a recommendation how much of a specific infant nutrition product that the infant should consume.

Any reference to prior art documents in this specification is not to be considered an admission that such prior art is widely known or forms part of the common general knowledge in the field.

The objective of the present invention was to enrich or improve the state of the art and in particular to provide a method, a system or device that is able to provide a personalized nutrition recommendation for an infant. This recommendation should be based on infant specific information to determine how much of a specific infant nutrition product that infant should consume, or to at least provide a useful alternative to existing solutions available in the art.

The inventors were surprised to see that the objective of the present invention could be achieved by the subject matter of the independent claims. The dependent claims further develop the idea of the present invention.

Accordingly, the present invention provides a method of preparing a personalized nutrition recommendation for an infant, the method comprising collecting infant-specific information, analyzing the collected information, and providing the nutrition recommendation based upon the analyzed collected information; wherein the nutrition recommendation comprises a recommendation how much of a nutrition product for infants the infant should consume. The child-specific information may comprise the sex of the child, the age of the child, and the weight of the child.

In a further aspect, the invention relates to computer implemented method comprising the method of the present invention.

Even further, the present invention relates to a system or a device implementing the method of the present invention.

As used in this specification, the words “comprises”, “comprising”, and similar words, are not to be interpreted in an exclusive or exhaustive sense. In other words, they are intended to mean “including, but not limited to”.

The present inventors have shown that the method of the present invention allows it to propose a suitable infant nutrition product and an optimal dosage for an infant based on information that is easily obtainable for a specific infant.

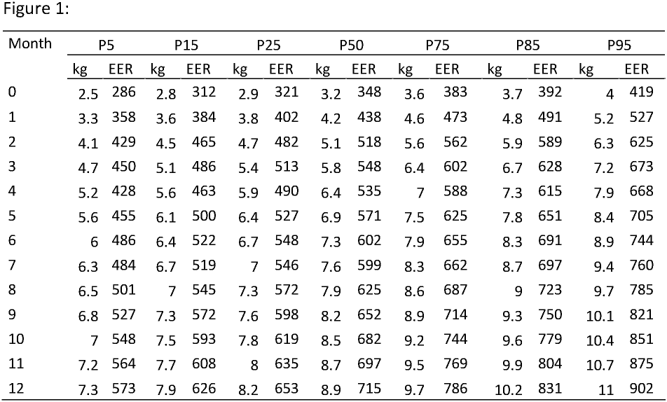

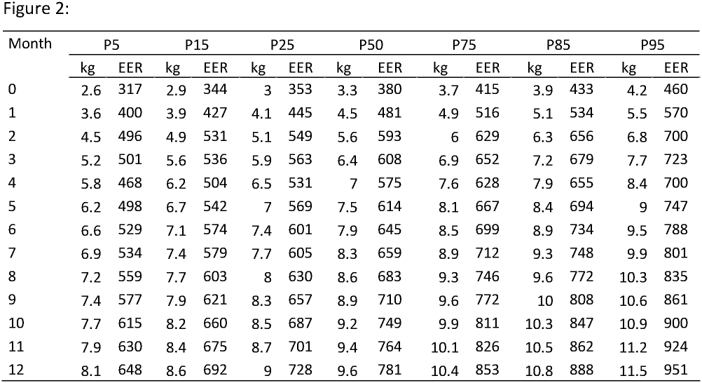

FIG. 1 shows an example of the Estimated Energy Requirement (EER) for girls, by percentile, as per the 2006 WHO Growth Standards. FIG. 2 shows an example of the Estimated Energy Requirement (EER) for boys, by percentile, as per the 2006 WHO Growth Standards.

Consequently, the present invention relates in part to a method of preparing a personalized nutrition recommendation for an infant, the method comprising collecting information, wherein the collected information comprises the sex of the child, the age of the child, and the weight of the child, analyzing the collected information, and providing the nutrition recommendation based upon the analyzed collected information; wherein the nutrition recommendation comprises a recommendation how much of a nutrition product for infants the infant should consume.

In the present specification, the following words are given a definition that must be taken into account when reading and interpreting the description, examples and claims.

Infant: According to the Commission Directive 91/321/EEC of 14 May 1991 on infant formula and follow-on formula, article 1.2 (a), the term “infants” means children under the age of 12 months. This definition is adopted in the present specification.

The term “nutritional product for infants” means foodstuffs intended for particular nutritional use by infants.

The term “infant formula” means foodstuffs intended for particular nutritional use by infants and satisfying by themselves the nutritional requirements of this category of persons. It has to be understood that infants can be fed solely with infant formula, or that the infant formula can be used by the carer as a complement of human milk.

The nutritional recommendation may be based on official guidance. For example, the World Health Organization has published in 2006 the WHO Child Growth Standards that might be used as a guidance. These standards can then be used to calculate energy requirements of infants 0-12 months of age, as per Butte et al. (2005, Public Health Nutrition: 8(7A), 953-967).

The nutritional recommendation may be based on the Estimated Energy Requirements (EER). The Estimated Energy Requirement (EER) is the average dietary energy intake that is predicted to maintain energy balance in healthy, normal weight individuals of a defined age, gender, weight, height, and level of physical activity consistent with good health. In infants, the EER may include the needs associated with growth at rates consistent with good health.

Further guidance on how to determine the EER may be obtained from WHO, Energy and Protein Requirements. WHO Tech Rep Ser no 724. Geneva; 1985; Food and Nutrition Board. Dietary reference intakes for energy, carbohydrate, fiber, fat, fatty acids, cholesterol, protein, and amino acids (macronutrients) have been published by the Institute of Medicine, Washington (DC): National Academy Press; 2005 and the FAO/WHO Geneva: WHO, 1985.

For example, the nutrition recommendation may be based on the on the Estimated Energy Requirement (EER) calculated based on the sex of the infant, the age of the infant, and the weight of the infant. For example, the EER may be determined based on body weight values from the 5^{th} to 95^{th} percentile of the Growth Chart Percentiles from the World Health Organization (WHO), for example available from WHO Multicentre Growth Reference Study Group. WHO Child Growth Standards: Length/height-for-age, weight-for-age, weight-for-length, weight-for-height and body mass index-for-age: Methods and development. Geneva: World Health Organization, 2006. The inventors have obtained very good results, when they used the EER determined based on body weight values from the 25^{th} to 75^{th} percentile

Determining the EER based on the body weight values from the 5^{th} to 95^{th} percentile of the Growth Chart Percentiles from the World Health Organization (WHO) has the advantage that the personalized nutrition recommendation is based on the infant’s individual growth status

In one embodiment of the present invention the EER is determined in accordance with FIG. 1 for girls and in accordance with FIG. 2 for boys.

Once the EER is determined, the EER is compared to the energy content of a nutrition product for infants to be consumed. The amount of the nutrition product for infants to be consumed be the specific infant may then be calculated accordingly. For example, the EER is compared to the energy content of the nutrition product for infants to be consumed, and the amount of the nutrition product for infants to be consumed per a specific time period is calculated accordingly. The time period to be considered may be per day or per consumption occasion, for example per nutrition products for infants serving.

Consequently, the method of the present invention allows it to determine the optimal amount of a nutrition product to be consumed by a particular infant.

Determining the optimal amount of a nutritional product to be consumed by an infant can be even more challenging, if the nutritional product for children is not the only source of nutrition the infant is consuming. This may be the case, for example, when the infant is partially breast-fed or when the infant already consumes other foods. For the purpose of the present invention, the term “food” shall mean in accordance with Codex Alimentarius any substance, whether processed, semi-processed or raw, which is intended for human consumption, and includes drink, chewing gum and any substance which has been used in the manufacture, preparation or treatment of “food” but does not include cosmetics or tobacco or substances used only as drugs.

In general, the more detailed the collected information is, the more carefully can the personalized nutrition recommendation can be calibrated.

For example, if the infant is consuming mother’s milk and/or other food, an estimate of the amount of mother’s milk and/or other food consumed by the infant per day can be determined, and the amount of the nutrition product for infants to be consumed per day may be calculated taking into account the amount of mother’s milk and/or other food consumed per day.

If the infant is consuming other foods in addition to the nutrition product, also these additional foods may be taken into account when preparing a personalized nutrition recommendation. Hence, the collected information may further comprise the amount of other nutrition consumed by the child per day, and the amount of the nutrition product for children to be consumed per day may be calculated taking into account the amount of other nutrition and mother’s milk consumed per day.

There are many other factors that will impact the nutritional needs and preferences of infants and their parents or caretakers. Such factors may be specific nutritional needs of the infant, for example in case of allergies or the prevalence of a certain allergy in the family. Such allergies or the risk to develop allergies can be taken into account when preparing a personalized nutrition recommendation for the infant.

Some infants may suffer from intolerances, for example lactose intolerance. Such intolerances can be taken into account when preparing a personalized nutrition recommendation for the infant.

Some infants may suffer from digestive discomforts. Also, such digestive discomforts can be taken into account when preparing a personalized nutrition recommendation for the infant. The digestive discomfort may be selected from the group consisting of gas, bloating, fussiness, reflux, colic, diarrhea, constipation, or combinations thereof.

The digestive discomfort may be a functional GI disorder, for example. Functional GI disorders are disorders of gut-brain interaction. It is a group of disorders classified by GI symptoms related to any combination of the following: motility disturbance, visceral hypersensitivity, altered mucosal and immune function, altered gut microbiota, and altered central nervous system processing. Functional GI disorders are for example defined in Gastroenterology. 2016;150:1257-1261, and The Rome IV Committees. Rome IV functional gastrointestinal disorders - disorders of gut-brain interaction. I. Raleigh, NC: The Rome Foundation; 2016.

Accordingly, the collected information may further comprise one or more elements of information selected from the group consisting of whether or not there is an increased risk of allergies in the family, whether or the child suffers from lactose intolerance, whether or not the child suffers from any digestive discomforts or if the child has any specific dietary preferences.

The inventors have found that it may be preferred if the method of the present invention is carried out as a two-step process. In a first step, the optimal nutritional product for infants will be determined based on the collected information. Once the optimal infant nutrition product has been identified, in a second step the optimal amount of the nutritional product that was identified in the first step will be determined based on the collected information. As a result, the method of the present invention will recommend an optimal amount of an optimal infant nutrition product for the specific infant that the information was collected from. Hence, in accordance with the present invention analyzing the collected information may comprise a first step to determine whether or not the infant requires a nutrition product for infants that takes into account specific medical or nutritional needs or preferences to determine an appropriate nutrition product for infants, and a second step to determine the amount of the appropriate nutrition product for infants that the infant should consume.

Nutritional preferences may be the nutritional preferences that the parents or carers have for the infant and may include, for example a preference for organic nutrition products for infants, locally produced nutrition products for infants, vegetarian nutrition products for infants, vegan nutrition products for infants, or combinations thereof.

In one embodiment of the method of the present invention, analyzing the collected information may comprise
a. a step to determine whether or not there is an increased risk of allergies in the family,
b. a step to determine if the infant is diagnosed with an allergy or intolerance, for example, selected from the group consisting of cow’s milk intolerance and lactose intolerance,
c. a step to determine if the infant suffers from digestive discomforts, for example, selected from the group consisting of gas, bloating, fussiness, reflux, colic, diarrhea, constipation, or combinations thereof,
d. a step to determine an appropriate nutrition product for infants based on the information collected above
e. a step to determine the estimated energy requirements (EER) of that infant based on their age, weight and gender
f. a step to determine the percentage of the daily energy intake that is not covered by mother’s milk consumption or the consumption of other nutrition, and
g. a step to calculate the optimum amount of the appropriate nutrition product for infants that should be consumed per day to fulfil their estimated energy requirements.

The method of the present invention may be a computer-implemented method. Hence, the method of the present invention may involve the use of a computer, computer network or other programmable apparatus, where one or more features are realised wholly or partly by means of a computer program. As such the computer implemented method of the present invention may be linked to a dispensing device so that the dispensing device used the recommendation how much of a nutrition product for infants the infant should consume that is produced by the method of the present invention to dispense a personalized portion or plurality of portions of a nutritional product for infants.

Hence, the computer-implemented method of the present invention may be used to control the internal functioning or operation of a computerized dispensing device, for example, a bulk dispensing device.

Accordingly, the method of the present invention may comprise the use of a database of available nutrition products for infants. The database may contain the nutritional information of each nutrition product, its intended application, and any further characteristics that may be relevant based on the information collected.

Hence, in the method of the present invention analyzing the collected information may involve the use of a database of available nutrition products for infants that contains the nutritional information of these nutrition products, their intended application and any further specificities that may become relevant when comparing the product information with the collected information, and the appropriate nutrition product is determined through comparing the collected nutritional requirements or preferences with the intended application of the nutrition products and any further specificities of the nutrition products and/or the optimum amount of a nutrition products for infants to be consumed is determined based on its nutritional information and the determined EER.

In accordance with the present invention the infant may be from 0-6 months old or from 7-12 months old. Typically, infants from 0-6 months old will consume the nutritional product for infants as only nutrition source, possibly in combination with mother’s milk.

Hence, the infant for which the method of the present invention is determining a personalized nutrition recommendation may be an infant that consumes the nutrition product for infants as the only nutrient source, optionally next to mothers’ milk.

Infants older than 6 months, for example from 7-12 months old, will already consume other foods as well.

The nutrition product for infants for which the method of the present invention recommends a dosage may be an infant formula.

Those skilled in the art will understand that they can freely combine all features of the present invention disclosed herein. Features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLES

### Method

### Step 1: Determining Appropriate Infant Nutrition Product

Data on the infant’s age, diagnosed allergies/familial allergy risk, digestive discomforts and personal preferences (organic etc.) are collected. This data is analysed against a specific database of infant nutrition products, and a recommendation for the product that best fits the infant’s needs is recommended. If this recommendation is provided within a computer-implemented method the data will be stored to provide an updated recommendation as the baby develops (i.e. to move from a product specific to 0-6 month olds, to a product relevant for 6-12 month olds). Step 2: Determining the appropriate serving size recommendation

Data on the infant’s gender, age and weight is collected. The following validated scientific algorithm is applied to calculate the infants estimated energy requirements (EER) (Butte et al (2005, Public Health Nutrition: 8(7A), 953-967))

$$\text{EER} \left[\text{kcal} / \text{day}\right] = 89 \text{*weight} \left[\text{kg}\right] - 100$$

The calculated EER are assigned to a gender-specific growth (weight-by-age) percentile (5^{th} to 95^{th}), as per the 2006 World Health Organisation (WHO) growth charts. In the event that the infant has multiple sources of calories (e.g. breastmilk, infant formula and/or foods) the proportion of kilocalories coming from breastmilk (using a question on of the total number of feeds how many are breastmilk) and the contribution from foods is calculated based on the age, using data from existing literature (Feeding Infants and Toddlers Study 2016; WHO. Infant and young child feeding, Geneva: 2009; Dewey KG. Pediatric Clinics of North America. 2001;48(1):87-104; Pan American Health Organisation (PAHO). Guiding principles for complementary feeding of the breastfed child 2003).The amount (volume) of the specific infant nutrition product (as determined in Step 1) needed to fulfil the child’s energy requirements is then calculated using the following formula:

$$\frac{E E R \left(a s c a l c u l a t e d a b o v e\right) ∗ 100}{E n e g y d e n s i t y o f r e c o m m e n d e d i n f a n t n u t r i t i o n p r o d u c t \left(\frac{k c a l}{100 m l}\right)}$$

The last step is to calculate the number of servings per day of the recommended infant nutrition product, considering the specific manufacturer instructions, using the following equation:

$$\frac{V o l u m e o f s p e c i f i c i n f a n t n u t r i t i o n p r o d u c t r e q u i r e d t o f u l f i l E E R}{R e c o m m e n d e d m a n u f a c t u r e r s e r v i n g s i z e}$$

This provides a personalised nutrition serving size for a specific infant nutrition product, in both volume and the number of servings per day required to fulfil the infants energy requirements. In the event that the personalised recommendation is linked to a connected device, this last step can be omitted. If this personalised serving size recommendation is provided within a computer-implemented method the data will be stored to provided an updated recommendation as the baby develops (i.e each month) as the babys energy requirements will increase with increasing age.

1. A method of preparing a personalized nutrition recommendation for an infant, the method comprising:
a. Collecting information, wherein the collected information comprises the sex of the child, the age of the child, and the weight of the child,
b. Analyzing the collected information, and
c. Providing the nutrition recommendation based upon the analyzed collected information;
wherein the nutrition recommendation comprises a recommendation how much of a nutrition product for infants the infant should consume.

2. The method in accordance with claim 1, wherein the nutrition recommendation is based on the on the Estimated Energy Requirement (EER) calculated based on the sex of the infant, the age of the infant, and the weight of the infant.

3. The method in accordance with claim 2, wherein the EER is determined based on body weight values from the 5^{th} to 95^{th} percentile of the Growth Chart Percentiles from the World Health Organization (WHO).

4. The method in accordance with claim 2, wherein the EER is compared to the energy content of the nutrition product for infants, and the amount of the nutrition product for infants to be consumed per a specific time period .

5. The method in accordance with claim 4, wherein, if the infant is consuming mother’s milk, an estimate of the amount of mother’s milk consumed by the infant per day is determined, and wherein the amount of the nutrition product for infants to be consumed per day is calculated taking into account the amount of mother’s milk consumed per day.

6. The method in accordance with claim 4, wherein the collected information further comprises the amount of other nutrition consumed by the infant per day, and wherein the amount of the nutrition product for infants to be consumed per day is calculated taking into account the amount of other nutrition and mother’s milk consumed per day.

7. The method in accordance with claim 1, wherein the collected information further comprises one or more elements of information selected from the group consisting of whether or not there is an increased risk of allergies in the family, whether or the child suffers from lactose intolerance, whether or not the child suffers from any digestive discomforts or if the child has any specific dietary preferences.

8. The method in accordance with claim 7, wherein the digestive discomforts is selected from the group consisting of gas, bloating, fussiness, reflux, colic, diarrhea, constipation, and combinations thereof.

9. Method in accordance with claim 1, wherein analyzing the collected information comprises a first step to determine whether or not the infant requires a nutrition product for infants that takes into account specific needs to determine an appropriate nutrition product for infants, and a second step to determine the amount of the appropriate nutrition product for infants that the infant should consume.

10. Method in accordance with claim 7, wherein analyzing the collected information comprises
a. a step to determine whether or not there is an increased risk of allergies in the family,
b. a step to determine if the infant is diagnosed with an allergy or intolerance, for example, selected from the group consisting of cow’s milk intolerance and lactose intolerance,
c. a step to determine if the infant suffers from digestive discomforts, for example, selected from the group consisting of gas, bloating, fussiness, reflux, colic, diarrhea, constipation, or combinations thereof,
d. a step to determine an appropriate nutrition product for infants based on the information collected above
e. a step to determine the estimated energy requirements (EER) of the infants based on their gender, age and weight
f. a step to determine the percentage of the daily energy intake that is not covered by mother’s milk consumption or the consumption of other nutrition and
g. a step to calculate the optimum amount of the appropriate nutrition product for infants that should be consumed per day to fulfil their estimated energy requirements.

11. The method in accordance with claim 1, wherein the method is a computer-implemented method.

12. The method in accordance with claim 9, wherein analyzing the collected information involves the use of a database of available nutrition products for infants that contains the nutritional information of these nutrition products, their intended application and any further specificities that may become relevant when comparing the product information with the collected information, and the appropriate nutrition product is determined through comparing the collected nutritional requirements or preferences with the intended application of the nutrition products and any further specificities of the nutrition products and/or the optimum amount of a nutrition products for infants to be consumed is determined based on its nutritional information and the determined EER.

13. The method in accordance with claim 1, wherein the infant is from 0 to 12 months old.

14. The method in accordance with claim 1, wherein the infant consumes the nutrition product for infants as the only nutrient source, optionally next to mothers’ milk or food.

15. The method in accordance with claim 1, wherein the nutrition product for infants is an infant formula.
